(19) United States
(12) Patent Application Publication
(10) Pub. No.: US 2016/0228630 A1
(43) Pub. Date: Aug. 11, 2016

(54) INTRACORPOREAL ARTIFICIAL LUNG

(71) Applicant: Michael Mirzoyan, Las Vegas, NV (US)

(72) Inventor: Michael Mirzoyan, Las Vegas, NV (US)

(21) Appl. No.: 14/619,446

(22) Filed: Feb. 11, 2015

(51) Int. Cl. A61M 1/32; A61M 39/24; A61M 39/08

(52) U.S. Cl. CPC A61M 1/32; A61M 39/08; A61M 39/24; A61M 2205/7545; A61M 2210/12; A61M 2205/0238; A61M 2205/04

(57) ABSTRACT

The present invention describes a new artificial lung inserted within the human chest. The principles governing functionality of this device are gravity dependent rotation and separation of the oxygen from circulatory system by gravity, different diameters of pores on the oxygen tube with angled channels in the vertical mesh septum and incorporation of one-way valves. Other advantages of the design: it does not contain porous fibers and its inherent resilience and capacitance may play decisive role in long-term management of patients with respiratory failure and substituting need for lung transplants. Moreover, the design has a relatively small prime blood volume and takes into consideration prevention of right ventricular strain by maintaining pulmonary arterial pressure within the physiologic range. It's placement within pulmonary circulation in parallel anastomosis is considered to be least stressful for the heart. The relative simplicity of the device to prior art is another striking advantage.

### FIELD OF THE INVENTION

The present invention relates to medical devices. More specifically, the invention is an artificial lung device that is safe, effective, and amenable within a patient's vascular structure.

### BACKGROUND OF THE INVENTION

The lung is the most important organ in the respiratory system. In the lung, venous blood is relieved of carbon dioxide and then oxygenated by air drawn through the trachea and bronchi into the alveoli.

In this regard, air travels from the mouth and nasal passage to the pharynx and the trachea. Two main bronchi, one on each side, extend from the trachea. The main bronchi divide into smaller bronchi, one for each of the five lobes. These further divide into a greater number of smaller bronchioles.

Additionally, there are about 50 to 80 terminal bronchioles in each lobe. Each of these divides into two respiratory bronchioles, which in turn divide to form 2 to 11 alveolar ducts. The alveolar sacs and alveoli arise from these ducts. The spaces between the alveolar sacs and alveoli are called atria.

Lung disease remains one of the major healthcare problems in the United States today. Two significant contributors to lung diseases are interstitial lung disease (ILD) and chronic obstructive pulmonary disease (COPD).

Interstitial Lung Disease (ILD) is a broad term applied to disorders of both known and unknown etiology characterized by fibrosis and inflammation. Examples of known etiologies for ILD include occupational exposures (silicosis, asbestosis, berylliosis, coal miner's pneumoconiosis and hard metal pneumoconiosis), infectious exposures (fungal disease and post-viral syndromes), systemic rheumatoid disorders (rheumatoid arthritis, systemic lupus erythematosis, Sjogren's syndrome, systemic sclerosis, dermatomyositis/polymyositis, mixed connective tissue disease and ankylosing spondilytis) and other miscellaneous causes (drug-induced pneumonitis, oxygen toxicity, radiation exposure, hypersensitivity pneumonitis and ARDS sequelae). Fibrotic/inflammatory interstitial lung disease of unknown etiology is termed idiopathic pulmonary fibrosis (IPF).

In contrast to ILD, chronic obstructive pulmonary disease is a persistent, irreversible condition that slowly progresses over time. COPD refers to the existence or co-existence of chronic bronchitis and emphysema and is characterized by obstructed airways, enlarged air spaces, destruction of lung parenchyma, occlusion of small airways, and reduced lung elasticity. When compared to normal, healthy lung function, patients with advanced stages of COPD are required to expend 10 to 20 times more effort to facilitate breathing.

General medical treatment for advanced ILD and COPD is the use of medications, mechanical ventilation, and the application of an extracorporeal membrane device (Artificial lung). The choice of treatment depends on the severity of the disease as well as the patient's response to prior therapies. Because of the high mortality rates for the lung diseases, however, each therapy has certain associated limitations which can cause the patient's discharge from the hospital and/or affect recovery.

Drug therapies to improve the patient's ailing condition are the least invasive and most readily deployed treatments of lung failure.

Multiple pharmacologic strategies have been developed since the 1960's, but despite therapeutic benefits, none of the examined treatments have demonstrated an ability to improve patient survival.

Mechanical ventilation is the most common therapy and serves to maintain respiratory function by rhythmically inducing a controlled flow of air into the lungs. In healthy persons, normal breathing consists of contracting the diaphragm to distend the lungs and create negative pressure between the atmosphere and the lungs, therefore forcing fresh air into the lungs. Following oxygen and carbon dioxide exchange, the diaphragm relaxes, compressing the lungs and forcing expiratory air into the external environment. Mechanical ventilation creates this effect but in an opposing manner; fresh air is driven into the lungs by positive pressure and expiratory air is pumped out of the lungs by negative pressure.

Mechanical ventilation treatment is associated with multiple shortcomings termed ventilator-induced lung injury, or VILI. VILI covers a range of detrimental effects to the lung that can postpone recovery, cause unfavorable outcomes, or even intensify preexisting injury.

The third clinical therapy that is often administered utilizes a membrane oxygenator and an accompanying flow circuit. The treatment is denoted as extracorporeal membrane life support (ECLS), often times referred to as extracorporeal membrane oxygenation (ECMO). An ECLS device processes patient blood by adding oxygen and removing carbon dioxide through fiber membrane technology, replicating the natural function of the lungs.

ECLS is employed under circumstances of severe, reversible respiratory failure, or to patients responding adversely to all advanced modes of mechanical ventilation. Operation of the circuit relies on a pump to draw blood from the vena cava, transport it through the membrane oxygenator, and return the blood either to the right atrium (venovenous bypass) or aorta (venoarterial bypass). Patients still receive mechanical ventilation while on ECLS; however, settings are reduced to minimize VILI as a result of the exchange of blood gases in the oxygenator. With less work required from the lungs, ECLS permits physiological complications to abate. The therapy can be applied for weeks barring complications.

Limitations of ECLS primarily arise from external circuitry and artificial blood contacting surfaces. To avert thrombosis within the circuit, patient blood is continuously anti-coagulated because bleeding is a major risk, whether internal (intracranial) or from cannula dislodgement. Patients are paralyzed and/or heavily sedated to minimize movement that could cause dislodgement, which creates a high risk scenario for decubiti's ulcers. Also, the continuous exposure of the blood to artificial surfaces causes platelets to adhere and/or alter function (thrombocytopenia), requiring the patient to receive multiple platelet transfusions. In addition to this, the ECLS circuit must also be constantly monitored for mechanical failures such as tubing degradation, oxygenator or pump failure, and presence of gaseous emboli or clot formation. Other noted complications include sepsis and renal failure. Finally, ECLS requires a multidisciplinary team to provide care. Overall cost of the procedure, including compensation for medical personnel, as well as restriction to major medical centers are further limitations to providing this therapy. The high overall cost of the procedure coupled with the fact that only major medical centers are outfitted to properly perform it hinder the widespread use of this therapy.

Artificial lung devises placed within the patient's vasculature and based on intravascular respiratory support therapy can be used as an additional and/or alternative approach to treating advanced (end stage) ILD and COPD. Such devices typically use fiber membrane technology to achieve exchange of gases (CO2 and O2), oxygenating blood and removing carbon dioxide. In most situations, carbon dioxide removal is a primary goal of intravascular devices since sufficient oxygenation levels can be attained in the clinical setting through nasal oxygen or specific ventilation modes.

The ability of intravascular respiratory support devices to facilitate carbon dioxide removal from the circulation provides an advantage over sole mechanical ventilation strategies. By regulating hypercapnia, or elevated carbon dioxide levels in the blood, intravascular respiratory therapy allows ventilation at lower tidal volumes and pressures and thereby eliminates the deleterious effects that often develop with mechanical ventilation. Decreasing the intensity of the mechanical ventilation has been shown to improve mortality rates. In addition, the lung tissue affected by disease experiences a lower workload since the device itself is performing partial respiratory function. The reduced workload allows the injured tissue to rest and may improve tissue recovery.

There have been numerous efforts in the past 40 years to achieve artificial lung function. Unfortunately, no new innovative respiratory assistance therapy has been developed for patients with severe, life-threatening lung disease. This is largely due to inadequate knowledge of pulmonary pathophysiology, a lack of emerging therapies, and insufficient mechanisms for providing intermediate to long-term respiratory support. The lack of adequate technology for respiratory support for patients with deteriorating lung function, in particular, has had profound effects on the quality of life for this increasingly large segment of the population.

Due to the reasons discussed above, there has been great interest in developing an artificial means for accomplishing physiological exchange of gases (CO2 and O2) directly to the circulating blood and bypassing the diseased lungs. While previous attempts have shown some levels of success, the excessive cost and limited results have proven the method unsuccessful.

Therefore, a serious need exists for new technologies and therapeutic approaches that can provide intermediate to long-term respiratory support for patients suffering from severe pulmonary failure. The need for efficient and inexpensive technology that can achieve sustained exchange of gases (CO2 and O2) in the blood while bypassing the diseased lung, and without resorting to chronic ventilation, remains paramount.

### SUMMARY OF THE INVENTION

In contrast to the current artificial lungs commonly used, this invention is a vastly improved version.

The general purpose of the present invention, which will be described subsequently in greater detail, is to provide a new and improved intra-corporeal gas exchange device without the disadvantages discussed above.

The invention provides an artificial lung device, including an inner sphere where pulmonary blood and oxygen mix. This inner sphere has about 200 to 400 ml internal capacity. A vertical mesh chamber septum extends from the bottom of the working chamber and divides the inner sphere into two chambers of equal volume: a blood chamber and an oxygen chamber.

The system also includes a pulmonary blood tube, which is situated in the lower ⅓ of the inner chamber. The pulmonary blood tube is internally separated by an impermeable septum.

The system also includes an oxygen tube situated in the lower ⅓ of the working chamber, across the pulmonary blood tube. The end of the oxygen tube is not porous, which prevents oxygen bubbles from crossing the vertical septum and entering the blood chamber. The system also includes a one-way valve, which open towards the oxygen chamber.

The system also includes an oval shaped fenestrated external cover outside of the working chamber to protect the inner sphere from external compression by organs such as the diaphragm and mediastinum. The external cover is fenestrated by numerous larger holes, e.g. 4-5 mm ID, which will allow the gas or occasional blood spills from the working chamber, to escape into the chest.

The system also includes a horizontal septum fenestrated by 4-5 mm (ID) holes. This septum is situated in the upper ⅓ of the working chamber and at the upper end of the vertical inter chamber septum.

The system also includes a one-way valve with external diameter of 2 to 5 cm, situated around the oxygen hose at the entrance/exit of the oxygen hose to/from the chest. This one-way valve opens toward the outside of the chest.

The system also includes two symmetrically situated hermetic bearings, each located around the blood and oxygen tubes exactly across from each other, incorporated in the wall of the working chamber.

The devices of this invention are safe in operation, effective, clinically acceptable, and amenable to easy insertion. In general, the increased gas exchange efficiency (both CO2 removal and O2 exchange) of the devices and systems of the present invention allow the devices and systems to be fabricated with relatively small outer diameters.

The devices of the present invention are primarily made of plastic and several light metal parts which are designed for placement in the right and/or left hemi-thorax, i.e. right and/or left chest.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages, and the specific objects attained by its uses, reference should be had to the accompanying drawings and descriptive matter in which there are illustrated preferred embodiments of the invention.

### BRIEF DESCRIPTION OF THE FIGURES

The invention will be better understood and objects other than those set forth above will become apparent when consideration is given to the following detailed description thereof. Such description makes reference to the annexed drawings wherein:

FIG. 1 illustrates embodiment of an artificial lung device of the present invention.

FIG. 2 illustrates a vertical cross-section of a inter chamber septum.

FIG. 3 illustrates a vertical cross-section of a pulmonary blood tube A and an oxygen tube B.

### DETAILED DESCRIPTION OF THE INVENTION

In the following detailed description, reference is made to the accompanying figures which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description is, therefore, not to be taken in a limiting sense, and the scope of the present invention is defined by the appended description and their equivalents.

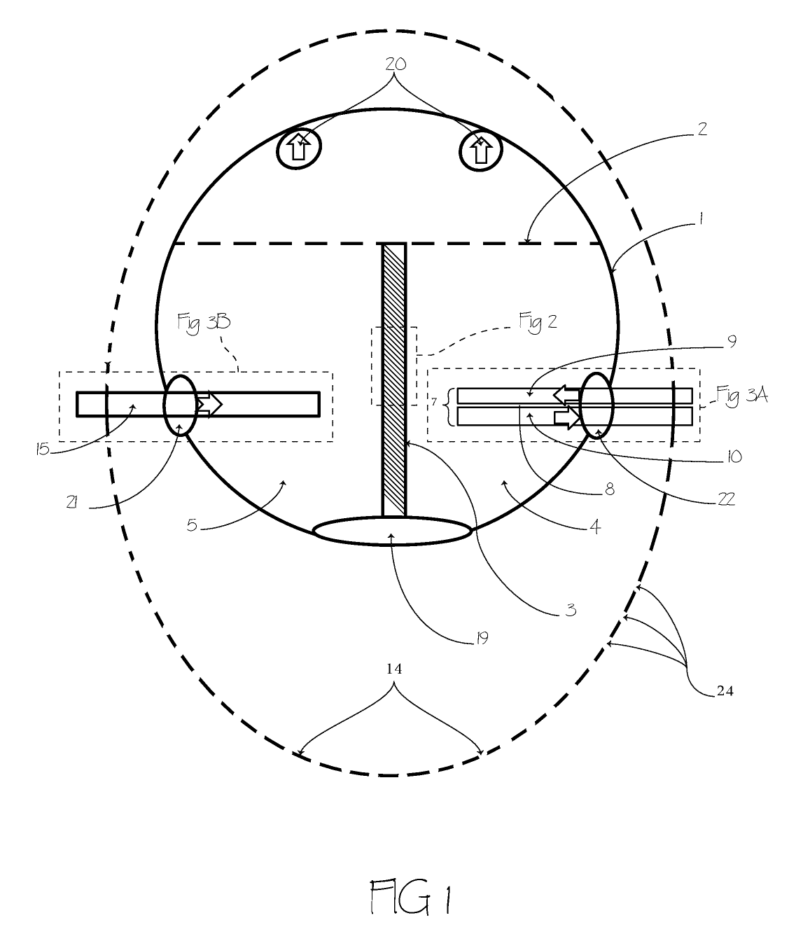

The device shown in FIG. 1 illustrates embodiment of an artificial lung device of the present invention. It describes a working chamber (inner sphere) 1 with 8-9 cm internal diameter and up to 400 ml, is the place where the pulmonary blood and oxygen mix. A ¾th of the working chamber (up to the horizontal septum 2) is prefilled with sterile crystalloid solution (e.g. normal saline) prior to employment of the device.

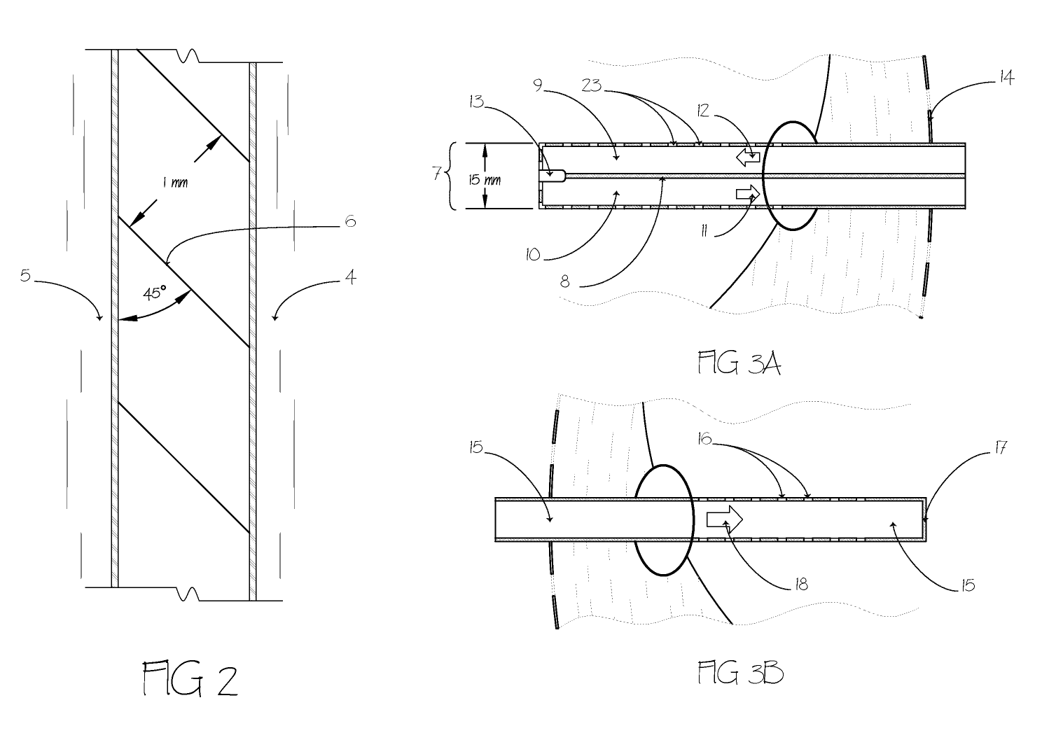

Another preferred embodiment of the present invention FIG. 2 provides a vertical inter chamber septum 3, about 0.5 to 1 cm thick, that separates the blood chamber 4 from the oxygen chamber 5 and extends from the bottom of the working chamber to ¾ of the height of the working chamber. The vertical inter chamber septum is fenestrated by 1 mm internal diameter (ID) channels 6, coursing at a 45 degree angle to the vertical axis of the septum, with the higher points of the channels open to the oxygen chamber 5 and the lower points of the channels open to the blood chamber 4.

Another embodiment of the present device FIG. 1 and 3A provides a pulmonary blood tube 7 which is situated in the lower ⅓ of the working chamber with an internal diameter of 1.5 cm. It is to be noted that the minimal diameter necessary to assure that the pulmonary arterial portion of the pulmonary blood tube has a sufficient diameter for the pulmonary arterial pressure must not exceed 15 mm Hg during systole.

In this design the pulmonary blood tube is separated internally by an impermeable septum 8 into two halves. The upper half of the tube is the pulmonary arterial portion 9, which carries deoxygenated blood from the right ventricle of the heart into the blood chamber 4. The lower half is the pulmonary venous portion 10 that carries the oxygenated pulmonary venous blood from the blood chamber toward the left atrium of the heart. The proximal and distal points of the arterial and venous portions of the pulmonary blood tube contain one way valves 11 and 12 in their respective one-way directions.

In another embodiment the septum inside the pulmonary blood tube is incomplete, i.e. it ends with a gap 13 at the end of the septum and the diameter of this gap must be close to the diameter of the arterial portion of the tube. The communication provided by this gap at the end of the septum in the pulmonary blood tube may elicit a desirable venturi effect during systole, when pulmonary arterial blood is rushed through this gap inside the pulmonary venous portion, pulling oxygenated blood from the working chamber around the pulmonary blood tube via pores. During diastole, oxygenated blood will return to the heart via pores in the venous portion of the pulmonary blood tube 10, facilitated by the gravity effect of the blood weight above the pulmonary blood tube in conjunction with constant movement of the blood within the working chamber 1 produced by continuous oxygen flow in the neighboring oxygen chamber 5. The entire perimeter of the pulmonary blood tube inside the working chamber (including arterial and venous portions, as well as the end of the tube) has 2 mm ID pores 23. Note: there are no pores on the pulmonary blood tube outside the working chamber 1. The pulmonary blood tube exits the working chamber, and after passing the external cover 14, transitions into 2 synthetic vascular grafts (Not shown): the upper vascular graft is attached to the native pulmonary artery, and the lower graft is to the native pulmonary veins.

Another embodiment of the present device FIG. 3B provides an Oxygen (O2) tube 15 which is from 0.5 to 1.5 cm (ID) situated in the lower ⅓ of the working chamber, across from the pulmonary blood tube. The oxygen tube is fenestrated with numerous 2 mm (ID) pores 16. The end 17 of the oxygen tube must not contain pores, since that surface points toward the vertical inter chamber septum. At the beginning of the oxygen tube, inside the working chamber, there is one-way valve 18 which opens toward the oxygen chamber. The absence of pores at the end of the oxygen tube serves as an additional safety feature to prevent oxygen bubbles from bombarding the vertical septum and entering the blood chamber. Outside of the working chamber 1 and after passing the external cover 14, the oxygen tube transitions into a synthetic non compressible hose which leaves the chest and connected to the oxygen source. To insure the sterility of delivered oxygen from the oxygen source, a high efficiency particulate filter (HEPA) should be employed before oxygen from the oxygen source enters the oxygen hose. Continuous flow of oxygen at 4-6 L/min should suffice to provide optimal gas exchange with respect to oxygenation and ventilation of the pulmonary arterial blood entering the working chamber.

In preferred embodiment of this device, at the bottom of the vertical septum 3, and in the wall of the working chamber, a weight 19 in order to prevent the working chamber full of blood to turn upside down. The goal of this design is to make the lower half wall of the working chamber heavier than the upper half, which could also be achieved by making the lower half of the working chamber of metal and the upper half of plastic. Advantageously this weight allows the working chamber to gravitate around its horizontal axis formed by blood and oxygen tubes, and keeping the working chamber in the gravity dependent patient's position at upright, semi-upright, supine, prone and upside down .

The direction related to the application of this device said that the person must not lie down on his/her side because the design lacks the lateral gravitational movement of the working chamber. During sleep, the recipient of the device must have special bed restraints to prevent the patient from turning on their side. The description of bed restraints is simple and will be provided upon request. In addition, as a solution to prevent sideways bending of the recipient while she/he is sitting or standing, wearing a rigid thoracolumbar corset during daily activities is strongly recommended. In some cases, a provision of unilateral or bilateral orthopedic thoracolumbar fixation may be considered to accompany surgical installation of the device into a patient's chest. All described one-way valves in this design must have the least resistance to opening pressures.

Another embodiment of the present invention is provides a horizontal 1-2 mm septum 2 fenestrated by 4-5 mm holes. This septum is situated in the upper ⅓ of the working chamber 1 above the upper end of the vertical inter chamber septum 3. The horizontal septum will restrain blood movement inside the working chamber when the recipient of the device is in motion, and may play a role in a mechanical defoaming of the blood when the foamed blood goes up inside the oxygen chamber 5.

In another embodiment of the device comprises two one-way valves 20 situated in the roof of the working chamber to vent the gas (CO2 and O2) outside of the working chamber. In another embodiment there are two symmetrically situated hermetic bearings 21 and 22, each of which is located around the blood 7 and oxygen 15 tubes exactly across each other. Each of these bearings is incorporated into the wall of the working chamber. These bearings form a horizontal axis around the blood and oxygen tubes. The blood and oxygen tubes run inside the bearings, and are sealed and fixed statically (hermetically) with inner portion of bearings. Because the working chamber has a heavier lower half, gravitational rotational movement will occur around the blood and oxygen tubes. This will keep the device in a gravity dependent position constantly.

There is an oval shaped fenestrated external cover 14 outside of the working chamber (e.g. 16 cm/height×16 cm/breadth×13cm/length or 14 cm×14 cm×11 cm, it depends on the diameter of the working chamber, e.g. 9 vs 8 cm etc., in order to allow the working chamber to make a 360 degrees unrestricted move inside the external cover). The function of the external cover is to protect the working chamber from external compression/impingement by intra thoracic organs such as the diaphragm and mediastinum. The gap between the external cover and the working chamber varies from 0.5 to 2 cm in their closest proximity to each other. This distance between the external cover and the working chamber makes possible an unimpeded 360 degrees gravitational rotation of the working chamber inside the external cover. The external cover is fenestrated by numerous larger holes 24, e.g. 4-5 mm ID, which allow gas (CO2 and O2) or occasional spills of blood from the working chamber to escape into the chest. The oxygen and blood tubes are fixed statically inside the external cover when they pass through it.

Another preferred embodiment of said invention includes a rectangular plate fixed on the back of the external cover with 4 to 8 screw points for a screw fixation of the plate to the posterior ribs inside the chest, positioning the working chamber at the level of the heart. The purpose of this plate is to provide the means of attachment of the device inside the chest.

A one-way valve with external diameter of 2 to 5 cm is situated around the oxygen hose at the entrance/exit of the oxygen hose to/from the chest. This one-way valve opens toward outside of the chest to release pressure that builds inside of the chest and to prevent ambient air from entering the chest.

The present device is coated with anti-adhesive film/surface like Teflon to prevent blood from fibrin/thrombosis formation within the device. As with any vascular prosthetic device, patients must be placed on a long-term anticoagulation regimen after receiving this device. In order to keep the working chamber free of blood clots and fibrin debris, it may be necessary to instill periodically (e.g. daily, every 3 days, weekly etc.) a small amount of thrombolytic agent such as Alteplase through a special port on the oxygen hose outside of the chest to prevent formation or degrade already formed fibrin/blood clots inside the working chamber.

Preferably, the device disclosed in the aforementioned disclosure could be made of plastic or light metal which does not restrict or limit the scope of invention, which allows the person skilled in the art in any future modification of related art.

The present invention will preferably be used inside of the body. Altogether the device is designed for placement in the right and/or left hemi-thorax, i.e. right and/or left chest. This device or its modifications could also be used as an artificial lung intra- corporeally, i.e. inside the human body or extra-corporeally, i.e. outside the human body.

The aforementioned dimensions are merely examples and could be customized to suit a patient's need should the device be required depending on the size of a chest cavity if the device to be employed inside the body.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

It is to be understood that the above description is intended to be illustrative, and not restrictive. For example, the above-discussed embodiments may be used in combination with each other. Many other embodiments will be apparent to those of skill in the art upon reviewing the above description.
1, An artificial lung device comprising:
An oval shaped fenestrated external cover protecting the inner sphere from external compression; A working chamber for mixing pulmonary blood and oxygen;
A gap between said external cover and said working chamber facilitating rotation of said working chamber inside said external cover space;
A horizontal septum located at the upper ⅓ of said working chamber;
A vertical inter chamber septum separating said working chamber into a blood chamber and an oxygen chamber;
A pulmonary blood tube located at the low ⅓ of said blood chamber having an impermeable septum separating said pulmonary blood tube in an upper pulmonary arterial portion carrying deoxygenated blood from the right ventricle of the heart into said blood chamber and a lower pulmonary venous portion carrying the oxygenated pulmonary venous blood from said blood chamber toward the left atrium of the heart;
An oxygen tube having at least one hole located at the low ⅓ of said oxygen chamber and connected through a high efficiency particulate filter to an oxygen source;
A weight on the bottom of said working chamber; and
A rectangular plate fixed on the back of said external cover.

2. The artificial lung of claim 1 device is coated with anti-adhesive film/surface like Teflon to prevent blood from fibrin/thrombosis formation within the device.

3. The artificial lung device of claim 1 wherein said external cover having at least one hole.

4. The artificial lung device of claim 1 wherein said horizontal septum having at least one hole located above said vertical inter chamber septum in said working chamber.

5. The artificial lung device of claim 1 wherein said vertical inter chamber septum extents from the bottom of said working chamber to the said horizontal septum and separates said blood and said oxygen chambers.

5. The artificial lung device of claim 1 wherein said vertical septum having channels coursing at a 45 degree angle to the vertical axis of the septum having the higher points of the channels open to said oxygen chamber and the lower points of said channels open to said blood chamber.

7. The artificial lung device of claim 1 wherein said pulmonary tube having a minimal internal diameter assuring the pulmonary arterial pressure below 15 mm Hg during systole.

8. The artificial lung device of claim 1 wherein said arterial and venous portions having one way valves in their respective one way directions.

9. The artificial lung device of claim 1 wherein said septum inside said pulmonary blood tube having a gap at the end.

10. The artificial lung device of claim 1 wherein said pulmonary blood tube having pores over the entire perimeter inside the working chamber. The artificial lung device of claim 1 wherein said oxygen tube attached to the oxygen hose with one-way valve located at the entrance of the oxygen hose to the chest and opened towards the outside the chest.

12. The artificial lung device of claim 1 wherein said pulmonary blood tube exiting said working chamber and said external cover attached by said upper pulmonary arterial portion to the native pulmonary artery, and said lower pulmonary venous portion to the native pulmonary veins.

13. The artificial lung device of claim 1 wherein said oxygen tube having pore over the entire perimeter except the end opened to said oxygen chamber.

14. The artificial lung device of claim 1 wherein said oxygen tube having a one-way valve opened toward said oxygen chamber.

15. The artificial lung device of claim 1 wherein said working chamber comprises two one-way valves situated in the roof of said working chamber.

16. The artificial lung device of claim 1 wherein said pulmonary blood tube and said oxygen tube having hermetic bearings incorporated into the wall said working chamber.

17. An artificial lung device comprising:
An oval shaped fenestrated external cover protecting an inner sphere from external compression;
A working chamber of 8-9 cm internal diameter having 200 to 400 ml internal capacity for pulmonary blood and oxygen mix;
A gap between said fenestrated external cover and said working chamber facilitating rotation of said working chamber inside said fenestrated external cover space;
A vertical inter chamber (mesh) 0.5-1.0 cm septum for separation of a blood chamber from an oxygen chamber;
A horizontal 1-2 mm septum having 4-5 mm holes situated in the upper ⅓ of said working chamber and at the level of upper end of said vertical inter chamber septum;
A pulmonary blood tube with 1.5 cm ID having pores over the entire perimeter comprising of an upper pulmonary arterial portion carrying deoxygenated blood from the right ventricle of the heart into said blood chamber and a lower pulmonary venous portion carrying the oxygenated pulmonary venous blood from said blood chamber toward the left atrium of the heart;
An oxygen (O2) tube about 0.5 to 1.5 cm (ID) connected through a high efficiency particulate filter to an oxygen source and having numerous 2 mm (ID) pores situated in the lower ⅓ of said working chamber, across said pulmonary blood tube;
A weight on the bottom of said vertical septum in the wall of said working chamber;
There are 2 one-way valves situated in the roof of the working chamber to vent the gas (CO2 and O2) outside of the working chamber; and
A rectangular plate fixed on the back of said external cover with 4 to 8 screw points for a screw fixation of said plate to the posterior ribs inside the chest, positioning the working chamber at the level of the heart.

18. The artificial lung device of claim 17 wherein said oval shaped fenestrated external cover is 16 cm/height×16 cm/breadth×13 cm/length or 14 cm×14 cm×11 cm.

19. The artificial lung device of claim 17 wherein said working chamber having 8-9 cm ID makes a 360 degrees unrestricted move inside the external cover.

20. The artificial lung device of claim 17 wherein a ¾th of said working chamber up to said horizontal septum is prefilled with sterile crystalloid solution prior to employment of the device.

21. The artificial lung device of claim 17 wherein said vertical inter chamber is fenestrated by 1 mm internal diameter (ID) channels, coursing at 45 degrees angle to the vertical axis of said vertical septum, with a higher point of said channels open to said oxygen chamber and the lower point of said channels open to said blood chamber.

22. The artificial lung device of claim 17 wherein said pulmonary blood tube separated inside by an incomplete impermeable septum having said gap at the end of the septum of said pulmonary blood tube into the upper half of the tube is a pulmonary arterial portion and the lower half is a pulmonary venous portion.

23. The artificial lung device of claim 17 wherein said 0.5-1.0 cm septum separating equal spaces of said blood chamber and said oxygen chamber and extending from the bottom of said working chamber to ¾ of the height of the working chamber.

24. The artificial lung device of claim 17 wherein said upper half of the pulmonary blood tube carries the deoxygenated blood from the right ventricle of the heart into said blood chamber and said lower half of the pulmonary blood tube carries the oxygenated pulmonary venous blood from said blood chamber toward the left atrium of the heart.

25. The artificial lung device of claim 17 wherein the proximal and distal points of arterial and venous portions of said pulmonary blood tube contain one way valves in their respective one-way directions.

26. The artificial lung device of claim 17 wherein said gap at the end of the septum of said pulmonary blood tube may elicit a desirable Venturi effect during systole when pulmonary arterial blood is rushed through said gap inside the pulmonary venous portion, pulling oxygenated blood from the working chamber around said pulmonary blood tube via said pores 2 mm ID over the entire perimeter.

27. The artificial lung device of claim 17 wherein said pores in said venous portion of said pulmonary blood tube facilitate the returning of oxygenated blood to the heart during diastole by the gravity effect of the blood weight above said pulmonary blood tube in conjunction with constant movement of the blood within said working chamber produced by continuous oxygen flow in said neighboring oxygen chamber.

28. The artificial lung device of claim 17 wherein said pulmonary blood tube exits said working chamber and after passing the external cover the upper vascular graft is attached to the native pulmonary artery and the lower graft is to the native pulmonary veins.

29. The artificial lung device of claim 17 wherein said end of said oxygen tube must not contain pores since that surface points toward said vertical inter chamber septum for avoiding the oxygen bubbles bombarding said vertical septum and entering said blood chamber.

30. The artificial lung device of claim 17 wherein one-way valve opens toward the oxygen chamber opens at the beginning of said oxygen tube, inside the working chamber.

31. The artificial lung device of claim 17 wherein said oxygen tube transits into a synthetic no compressible hose leaving the chest and connected to the oxygen source outside of said working chamber and after passing said external cover.

32. The artificial lung device of claim 17 wherein a high efficiency particulate filter (HEPA) is employed before continuous flow of oxygen at 4-6 L/min from said oxygen source enters the oxygen hose insuring the sterility of a delivered oxygen.

33. The artificial lung device of claim 17 wherein said weight keeping said working chamber full of blood in constant gravity dependent position preventing the upside down turns.

34. The artificial lung device of claim 17 wherein the lower half wall of said working chamber heavier than the upper half allowing said working chamber to gravitate around it's horizontal axis formed by said blood and oxygen tubes, and keeping said working chamber in the gravity dependent position at all times while the patient is in upright, semi upright, supine, prone and upside down positions.

35. The artificial lung device of claim 17 wherein special beds restraints must prevent the recipient of the device from turning on the side during the sleep.

36. The artificial lung device of claim 17 wherein two symmetrically situated hermetic bearings located around said blood and oxygen tubes exactly across each other, incorporated in the wall of said working chamber.

37. The artificial lung device of claim 17 wherein said blood and oxygen tubes run inside said bearings, sealed and fixed statically (hermetically) with inner portion of said bearings.

38. The artificial lung device of claim 17 wherein gravitational rotational movement of said working chamber with its heavier lower half occurs around said blood and oxygen tubes maintaining constant gravity dependent position.

39. The artificial lung device of claim 17 wherein said gap between said external cover and said working chamber is 0.5 to 2 cm in their closest proximity making possible an unimpeded 360 degrees gravitational rotation of said working chamber inside said external cover.

40. The artificial lung device of claim 17 wherein said external cover having larger holes, about 4-5 mm ID allowing gas (CO2 and O2) or occasional blood spills escape, from said working chamber into the chest.

41. The artificial lung device of claim 17 wherein a one-way valve opened toward outside of the chest with external diameter about 2 to 5 cm, situated around the oxygen hose at the entrance/exit of the oxygen hose to/from the chest for releasing the pressure from building inside the chest and from preventing the ambient air entering inside the chest.

42. The artificial lung device of claim 17 wherein all surfaces of said device are coated with anti-adhesive film/surface like Teflon preventing fibrin/thrombosis formation within the device.

43. The artificial lung device of claim 17 w herein said device is periodically treated with agent like Alteplase through a special port on the oxygen hose outside the chest for degradation of fibrin/blood clots inside said working chamber.

44. The artificial lung device of claim 17 wherein said device itself could be made of plastic or the light metal.

45. The artificial lung device of claim 17 wherein the placement of said device is in the right and/or left hemithorax, i.e. right and/or left chest.

46. The artificial lung device of claim 17 wherein said device placed intra corporeally, i.e. inside the human body or extra corporeally, i.e. outside the human body.

47. The artificial lung device of claim 17 wherein all said one-way valves having the least resistance to opening pressures.
